(19) United States
(12) Patent Application Publication
(10) Pub. No.: US 2004/0030243 A1
(43) Pub. Date: Feb. 12, 2004

(54) Sensor device for detecting the changes in position and location of a proband in a neuronavigation system

(76) Inventors: Udo Warschewske, Berlin (DE); Arno Schleich, Berlin (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,740

(22) PCT Filed: May 11, 2001
(86) PCT No.: PCT/EP01/05402

(30) Foreign Application Priority Data
May 11, 2000 (DE) 10022937.9

(51) Int. Cl. A61B 5/05

(52) U.S. Cl. 600/424

(57) ABSTRACT

The invention relates to a sensor device for detecting changes in location and position of a proband in a neuronavigation system which is used to carry out and support surgical interventions. Said device can be fixed to the head of the proband and the output signals are supplied to a system computer as correction data. According to the invention, a shaped tooth rack for receiving a fixing agent and a sensor capsule with means for detachably fixing to the tooth rack are provided. The shaped tooth rack is fixed in the oral cavity of the proband with the fixing agent, the sensor capsule being oriented towards the outside in the direction of the cheek so that the oral cavity remains essentially free.

### DESCRIPTION

The invention relates to a sensor device for detecting changes in position and location of a proband in a neuronavigation system which is used to carry out and support surgical interventions, with said device being fixable to the head of the proband, and the output signals being supplied to a system computer as correction data, according to the preamble of claim 1.

From PCT WO96/08209, a neuronavigation system, as well as a tracking means, is previously known for medical applications. Therein, an image database for pre-surgically taken nuclear magnetic resonance or computerized tomography shots is present, and a personal computer or control computer is in communication with a monitor for processing and representing image data. A tracking means serves for determining the current position of an instrument and for deriving representations of the patient's anatomy using the shots stored in the image database. Moreover, means for extracting anatomic structures from source data records of the presurgical shots, and making these structures available in the form of 3D image data records that can be visualized, is provided there. A transmitter generates a magnetic field in the navigation surrounding, with the pointer navigation instrument being provided with a magnetic field sensor. The position of the instrument can be detected by correspondingly determining a field intensity.

For determining a reference position, a further sensor is provided which is fixed by means of a clamp to the patient's head in the region of the nose. Such a kind of fixing of a correction sensor that senses positional changes of the proband's head, however, is unreliable and is in particular then impeding when neurosurgical interventions or surgical measures are scheduled in the ear, nose and throat region.

In an earlier teaching originating from the Applicant, recourse is taken to a specific continuous field transmitter for carrying out and supporting surgical interventions, with an integral magnetic field sensor being provided in the pointer navigation instrument. Said transmitter for generating the continuous magnetic field of the tracking means is arranged on the operating table or on a thereon provided head rest, yet, outside of the operation field, thereby resulting in a fixed, reproducible positional relationship between the patient's organ to be navigated and the transmitter, independent of the location of the operation table in the room. The magnetic field sensor according to the already proposed teaching supplies signals for deriving the position and/or movement direction of the pointer navigation instrument, by means of the defined continuous magnetic field and its field orientation, with these signals being able to be both represented on a PC monitor and serving for controlling the reloading and updating processes of an image processing module.

It has likewise been proposed therein to arrange a further magnetic field sensor, with same being able to be fixed immediately to the proband, preferably to the head, so as to detect changes in location and position relative to the continuous field transmitter. The output signals of this second magnetic field sensor are supplied to the system computer as correction data, in order to fix a quasi dynamic system of coordinates or a system of coordinates variable with respect to the original change.

However, it has shown that fixing the additional sensor to the head of a proband or patient is not unproblematic, since a secure fixation becomes necessary and, on the other hand, the operating surgeon must not be impeded in his further activity by this additional sensor.

From the aforementioned, it is therefore the object of the invention to propose a sensor device for detecting changes in position and location of a proband in a neuronavigation system, which is used to carry out and support surgical interventions. The device to be created is intended to be able to be securely fixed to the proband's head without the operating surgeon being impeded in his work. Furthermore, the sensor device must be able to be pre-surgically fixed in an easy and effortless manner, and must not lead to unnecessarily high cost or expenditures, as far as the kind of fixation is concerned.

The solution of the inventive object ensues with a sensor device according to the features of claim 1, with the dependent claims comprising at least purposeful configurations and improvements.

The basic idea of the invention accordingly resides in a two-part sensor device comprising, for one, a shaped tooth rack and, for another, a sensor capsule. The tooth rack and sensor capsule are provided with means for detachably mounting to each other. The tooth rack itself serves for receiving an adhesive fixing compound, e.g. a usual two-component dental impression material. By means of this adhesive material, the tooth rack is fixed to a row of the patient's teeth within the oral cavity.

The sensor capsule can preferably be connected to a portion of the shaped tooth rack by means of a prismatic guide, in particular a flat or dovetail guide. Of course, there is also the possibility of attaching the sensor capsule to the shaped tooth rack in a usual way by screws or similarly in a force-transmitting manner.

In one exemplary embodiment, the sensor capsule itself has an essentially rectangular housing shape, with a guide means complementary to the tooth rack being formed on the longitudinal housing side, and an attachment means for a sensor cable being furthermore provided on the housing. This attachment means may be a moisture-proof plug-in connection interacting with an associated plug means on the cable end.

The tooth rack preferably has a slightly curved shape, which essentially corresponds to a portion of a row of teeth. The area between the inner circle and the outer circle has break-throughs or recesses serving to fix the adhesive fixing compound, so that a displacement of the tooth rack fixed to the row of teeth is precluded.

In one exemplary embodiment, the profile of the tooth rack is configured U-shaped, with one profile leg having a first minor thickness, and the other profile leg having a second major thickness. The profile leg of major thickness serves to receive the means for detachably mounting the sensor capsule. For instance, a groove may be realized in this profile leg of major thickness for receiving a dovetail or flat guide, i.e. a ridge correspondingly formed on the housing of the sensor capsule.

In its state of use, the device is fixed in the proband's oral cavity to a row of teeth by means of the aforementioned adhesive compound, with the profile leg of major thickness comprising the thereon mounted sensor capsule being oriented towards the outside of the row of teeth in the direction of the cheek. Thus, it is secured that the proband's oral cavity remains essentially free, so that in this case no restrictions whatsoever are given for the surgeon. In particular, no interferences with other medial apparatuses arise such as, for example, an anesthesia tube. The proximity of the device to the surgical field moreover ensures a high degree of accuracy through a minimization of error vectors.

The image registration for the neuronavigation may ensue already at the proband's free head movement, without any other means than the sensor device described.

The data obtained by the sensor device, e.g. magnetic field data, are correspondingly taken into account during the calculation of the position of the actual neuronavigation pointer instrument, and are supplied to a computer present in the system.

Apart from the described prismatic, flat or dovetail guides, also locking connections, i.e. so-called snap-in connections between the sensor capsule and the tooth rack are, of course, likewise imaginable without departing from the presented inventive principle.

For hygienic reasons, the tooth rack may be configured as a disposable part, whereby there is the possibility of manufacturing same of a sterilizable material for the purpose of multiple use.

In the following, the invention will be explained in more detail by means of an exemplary embodiment and with the aid of Figures.

Therein shows:

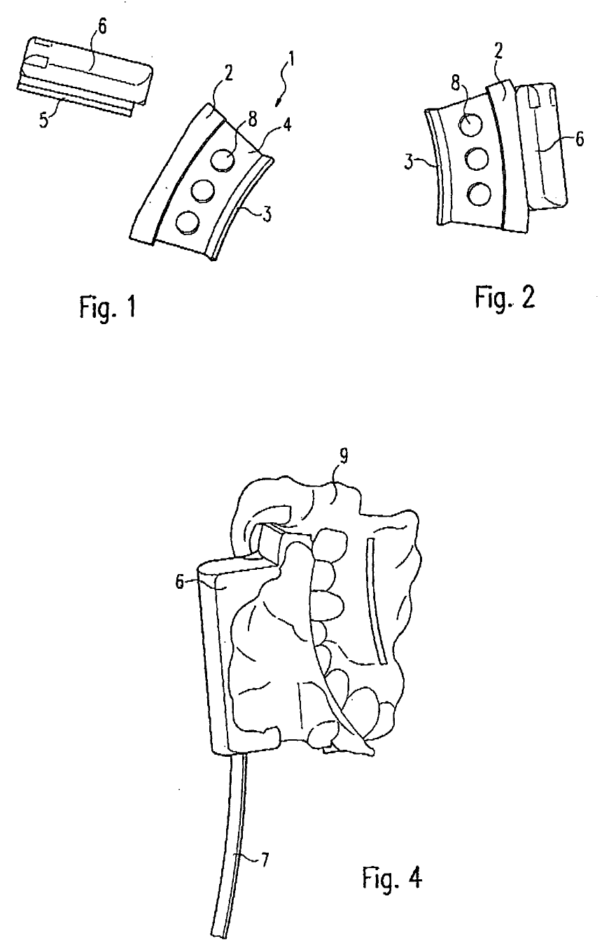

FIG. 1 a view of a sensor capsule and shaped tooth rack before being connected;

FIG. 2 a view of a sensor capsule that is in snap-in connection with the tooth rack via an appropriate guide;

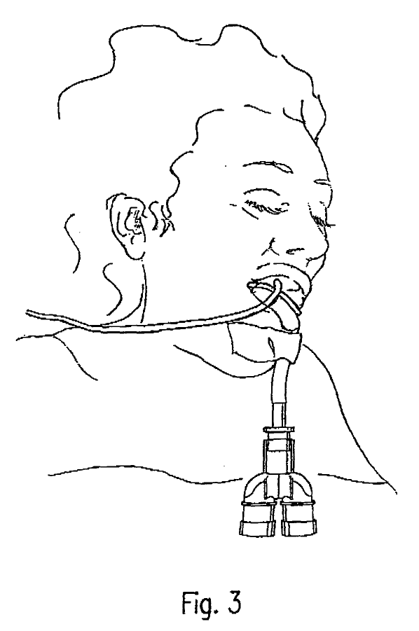

FIG. 3 a sensor device placed in a proband's oral cavity; and

FIG. 4 a representation of the sensor device including an adhesive fixing compound after removal from the proband's oral cavity.

According to the Figures, the sensor device comprises a profiled tooth rack 1 having one profile leg of major thickness 2 and one profile leg of minor thickness 3. In the embodiment shown, the portion 4 interconnecting the legs has circular breakthroughs 8 or bores that serve for an improved reception of the adhesive fixing compound 9 so that an undesired displacement of the device is precluded.

In the region of the leg of major thickness 2, a longitudinal groove is realized, which is complementary to a guide protrusion 5 on the housing 6 of the sensor capsule.

Said guide may, for example, be realized as a flat, prismatic or dovetail guide, with the guide protrusion 5 of course being also able to be realized on the leg of major thickness 2, and the groove complementary to same may be realized on the sensor capsule or the housing 6.

A plug-in socket not shown is provided on the housing 6 for mechanically attaching and electrically connecting a cable 7.

In the exemplary embodiment, the shape of the housing 6 is essentially rectangular and elongated, so that the sensor capsule finds place in the space between the row of teeth and the inner cheek area.

The kind of fixing the sensor device is illustrated in FIG. 3, whereby it also becomes obvious that the oral cavity remains free, e.g. for receiving a tube. The tooth rack 1 exhibits a slightly curved shape that approximately corresponds to the course of a portion of a row of teeth.

In its state of use, the tooth rack 1 is fixed to an inner tooth side of the proband by means of the adhesive compound 9, with the profile leg of major thickness 2 including the thereon mounted sensor capsule being oriented towards the outside of the row of teeth in the direction of the cheek. As the adhesive compound 9, two-component dental impression material is preferably used.

The tooth rack, which is detachable from the sensor capsule, may be manufactured in a particularly low-priced form as a disposable part, or may be manufactured of a sterilizable material for multiple use.

Altogether, it is achieved with the presented invention to keep the patient's head movable, a fact which entails surgical advantages as compared to the hitherto necessary rigid fixation. In addition, a correction data acquisition may take place by the simple and secure fixation of the sensor in the proband's oral cavity, so that changes in position and location of the head may be registered online without problems, and can be taken into account when determining the position of the pointer instrument.

It is within the meaning of the invention that the profile shape of the tooth rack may, as well, deviate from a U-shape, as long as same may still be fixed securely adhering to a row of teeth. The placement of the sensor device by means of the tooth rack and the rapidly setting impression material takes place at the time of the patient's preparation in the operating room after the patient's intubation.

List of reference numerals

1 tooth rack

2 leg of major thickness

3 leg of minor thickness

4 connecting portion of the legs

5 guide protrusion

6 housing

7 cable

8 breakthroughs in connecting portion

9 adhesive compound

1. A sensor device for detecting changes in position and location of a proband in a neuronavigation system, which is used to carry out and support surgical interventions, with said device being fixable to the head of the proband, and the output signals being supplied to a system computer as correction data, comprising
a curved tooth rack for receiving an adhesive fixing compound, and
a sensor capsule including means for detachably mounting to the tooth rack,
characterized in that
the area between the inner circle and the outer circle has breakthroughs or recesses serving for fixing the adhesive fixing compound, the tooth rack has an essentially U-shaped profile, with one profile leg having a first minor thickness, and the other profile leg having a second major thickness, and on the profile leg of major thickness, the means for detachably mounting the sensor capsule being formed, and that in its state of use, the tooth rack is fixed in the proband's oral cavity to a row of teeth by means of the adhesive compound in such a manner that the profile leg of major thickness including the thereon mounted sensor capsule is oriented towards the outside of the row of teeth in the direction of the cheek, so as to keep the oral cavity essentially free.

2. The device according to claim 1,
characterized in that
the means for detachably mounting are configured as prismatic, in particular flat or dovetail guide.

3. The device according to claim 1 or 2,
characterized in that
the sensor capsule has an essentially rectangular housing shape, with the guide means complementary to the tooth rack being realized on the longitudinal housing side, and furthermore, an attachment means for a sensor cable being provided on the housing.

4. The device according to any one of the preceding claims,
characterized in that
the tooth rack is realized as a disposable part or is manufactured of a sterilizable material for multiple use.
